(19) United States
(12) Patent Application Publication
(10) Pub. No.: US 2013/0137766 A1
(43) Pub. Date: May 30, 2013

(54) Compound Used to Prevent Diseases Caused by Aquaporin Deficiency

(71) Applicant: Fu-Jen Catholic University, New Taipei City (TW)

(72) Inventor: Chi-Feng Hung, Taipei City (TW)

(73) Assignee: FU-JEN CATHOLIC UNIVERSITY, New Taipei City (TW)

(21) Appl. No.: 13/743,730

(22) Filed: Jan. 17, 2013

Related U.S. Application Data
(62) Division of application No. 13/162,038, filed on Jun. 16, 2011.

(30) Foreign Application Priority Data
Nov. 23, 2010 (TW) 099140292

(51) Int. Cl. A61K 31/215; A61K 31/19

(52) U.S. Cl. CPC A61K 31/215; A61K 31/19
USPC 514/529; 514/557

(57) ABSTRACT

A compound used to prevent diseases caused by aquaporin deficiency, which is 18β-Glycyrrhetinic acid derivative. Said compound can not only prevent diseases caused aquaporin deficiency, but be able to prevent aquaporin (AQP) production and enhance skin function. Since AQPs have many advantages in skin cells, e.g. promoting water and glycerine molecular transportation, increasing skin elasticity and cuticle moisture, increasing the cell proliferation and cell migration, aquaporin can promote skin bather function and wound cicatrization. Therefore, said compound can be applied potentially as a medicinal cosmetic in skin medicine cosmetology, or as a new medical composition to treat diseases caused by AQP abnormality, such as urine concentration defect, wound healing slow down, corneal re-epithelialization slow down and etc.

### CROSS-REFERENCE TO RELATED APPLICATIONS

This application claims priority to, and is a Divisional of, U.S. patent application Ser. No. 13/162,038, filed on Jun. 16, 2011, now pending, which claims priority from Taiwan Patent Application No. 099140292, filed on Nov. 23, 2010, both of which are hereby incorporated by reference in their entirety.

Although incorporated by reference in its entirety, no arguments or 16 disclaimers made in the parent application apply to this divisional application.

Any disclaimer that may have occurred during the prosecution of the above-referenced application(s) is hereby expressly rescinded. Consequently, the Patent Office is asked to review the new set of claims in view of all of the prior art of record and any search that the Office deems appropriate.

### BACKGROUND OF THE INVENTION

1. Field of the Invention

The present invention relates to a compound used to prevent diseases caused by aquaporin deficiency, and the compound as shown in FIG. 1 is a 18β-glycyrrhetinic acid derivative. The concept of such compound includes any salts, solvates, or pharmacologically-functional derivatives of such compound.

2. Description of the Prior Art

Aquaporins, also called AQPs, are small (˜30 kDa), integrated membrane proteins. Thus far, 13 aquaporins were discovered (AQP-0˜12), and are classified into three groups according to their functions: aquaporins in the first group are responsible for water transport and include AQP-0, 1, 2, 4, 5, 6, and 8 (Verkman, 2005).

The second group, aquaglyceroporin, includes AQP-3, 7, 9, and 10. In addition to water transport, aquaglyceroporins also transport small molecules, e.g. glycerin and urea, etc. (Verkman, 2005).

AQP-11 and 12 belong to the third group, superaquaporin (Krane and Goldstein, 2007), and their functions remain unclear.

It was evidenced that several different aquaporins are expressed in mammalian skin. For example, AQP-1 is expressed at cell plasma membranes in fetal and newborn dermis; AQP5, on the other hand, is expressed in human sweat glands; and AQP-7 is expressed in the adipocytes located in subcutaneous tissue. AQP-3 is the best understood aquaporin and was initially found at the basal membrane in the epidermis in rat skin. In humans, AQP-3 is mainly expressed at the basal membrane in the epidermis (Hara-Chikuma and Verkman, 2008). In addition, Cao et al. demonstrated that AQP-3 is also expressed in the fibroblasts (Cao et al., 2006). AQP-3 facilitates water and glycerol transport in skin. Hence, it is involved in stratum corneum hydration. Literature has indicated that mice lacking AQP-3 manifest reduced stratum corneum hydration, premature aging, and lack of skin elasticity. These mice also exhibit significantly impaired epidermal and stratum corneum hydration (Hara-Chikuma and Verkman, 2008).

Glycyrrhizin is one of the major compositions of Glycyrrhiza species, and according to previous literature, glycyrrhizin has anti-inflammatory, anti-viral, and anti-allergic effects; and can inhibit prostaglandin secretion from macrophages. Furthermore, glycyrrhetinic acid is a metabolite of glycyrrhizin and an aglycon monomer; it also exhibits the same function as glycyrrhizin. In clinical skin care, glycyrrhetinic acid was used as an herb to treat various skin diseases, e.g. Dermatitis, Eczema, Pruritus, and Cysts, etc. The skin is a very important first line of defense system for the human body and provides protection against pathogen invasion; and stratum corneum plays an important role in the formation of the effective permeability barrier. Stratum corneum hydration is closely related with skin health and its normal physiological functions. Other factors that are involved in skin health are environmental humidity, skin structure, and the concentration of natural moisturizing factors, etc.

Application of glycyrrhetinic acid in skin care has been widely reported, and previous studies have also demonstrated its bioactivity. The effects of glycyrrhetinic acid on the activity and function of AQP-3 (e.g. wound healing), however, has not yet been clarified.

Given the above, after years of painstaking research and taking the novel applications of said derivative of 18β-Glycyrrhetinic acid in medicine cosmetology and diseases caused by aquaporin deficiency into consideration, the inventor(s) have developed a compound which can be used to prevent diseases caused by aquaporin deficiency, and said compound is a derivative of 18β-Glycyrrhetinic acid which can promote AQP-3 expression and subsequently enhance the skin function.

### SUMMARY OF THE INVENTION

The present invention features a compound which can be used to prevent diseases caused by aquaporin deficiency. Said compound, as shown in FIG. 1, is a 18β-Glycyrrhetinic acid derivative, where R could be one of H, CH₃, CH(CH₃)₂, and CH₂Ph.

In one aspect, the present invention provides a compound which can increase the expression of AQP in various cells. Subsequently, such a compound facilitates the transportation of water and glycerol between dermis and epidermis, increases skin moisture, and enhances the moisture retention capacity of the skin, so as to let the skin contain more moisture and be more viable.

In another aspect, said compound can facilitate migration and proliferation of the keratinocytes and fibroblasts. Consequently, the effect of improving the healing of wounded skins can be achieved.

In another aspect, the present invention provides a medicinal composition which can be used to prevent diseases caused by aquaporin deficiency. Such medicinal composition comprises an 18β-Glycyrrhetinic acid derivative as shown in FIG. 1 and pharmaceutically-acceptable carriers that include diluents, fillers, binders, disintegrating agents, or lubricants.

The following detailed description of the invention will be better understood when read in conjunction with the appended drawings. However, the invention is not limited to the preferred embodiments shown.

### BRIEF DESCRIPTION OF THE DRAWINGS

In the drawings:

FIG. 1 shows the source and structure of the 18β-glycyrrhetinic acid derivative, where R could be one of H, CH₃, CH(CH₃)₂, and CH₂Ph.

FIG. 2 shows the correlation between the expression of AQP-3 in fibroblasts and the concentration of the 18β-glycyrrhetinic acid derivative.

FIG. 3 demonstrates the correlation between the proliferation of the fibroblasts, examined by the MTT assay, and the concentration of the 18β-glycyrrhetinic acid derivative.

FIG. 4 shows the correlation between the proliferation of the fibroblasts, examined by the Trypan blue exclusion assay, and the concentration of the 18β-glycyrrhetinic acid derivative.

FIG. 5 shows the microscopic examination results under different concentrations of 18β-glycyrrhetinic acid derivative-induced fibroblast proliferation.

FIG. 6 is the result of in vitro scratch Wound Healing assay and shows the correlation between the cell migration and the increase of time under different concentrations of the 18β-Glycyrrhetinic acid derivative.

FIG. 7 is the result of Electric Cell-Substrate Impedance Sensing (ECIS) and shows the correlation between the changes of the resistance, expressing the change of cell comparative density and the increase of time under different concentrations of the 18β-glycyrrhetinic acid derivative.

FIG. 8 shows the expression of AQP-3 in human keratinocytes increases with time under the treatment of 30 μM 18β-glycyrrhetinic acid derivative.

FIG. 9 shows the correlation between the expression of AQP-3 and different concentrations of the 18β-Glycyrrhetinic acid derivative in human keratinocytes.

FIG. 10 is the MTT assay results and shows the correlation between the concentration of the 18β-glycyrrhetinic acid derivative and human keratinocytes proliferation.

### DETAILED DESCRIPTION OF THE PREFERRED EMBODIMENT

The present invention will now be described more specifically with reference to the following embodiments, which are provided for the purpose of demonstration rather than limitations.

### Embodiment 1

### Preparation of the 18β-Glycyrrhetinic Acid Derivative

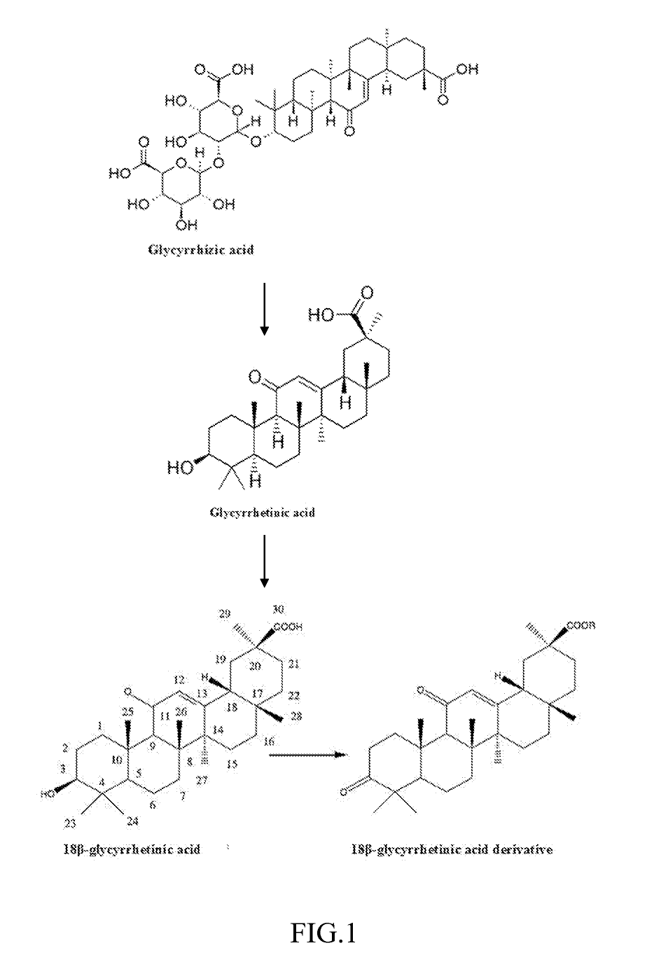

18β-glycyrrhetinic acid is a metabolite of glycyrrhizic acid (GL) and an aglycon monomer (FIG. 1). Several animal studies have indicated that 18β-glycyrrhetinic acid exhibits an anti-inflammatory effect (Maitraie et al., 2009). The 18β-glycyrrhetinic acid derivative used in the present invention was obtained by using a glycyrrhizic acid as a backbone and modifying the functional group at the C3 position of such glycyrrhizic acid, which is shown in FIG. 1. R in the 18β-glycyrrhetinic acid derivative could be one of H, CH₃, CH(CH₃)₂, and CH₂Ph.

### Embodiment 2

### Experiments about the Effects of the 18β-Glycyrrhetinic Acid Derivative on the Expression of AQP-3

1. Cell Culture

The research was operated by using human primary fibroblast and human keratinocyte line (HaCaT), both of which were isolated from human foreskin. The primary fibroblast and HaCaT were put in a Dulbecco's Modified Eagle Medium (DMEM) nutrient mixture that contains 10% fetal bovine serum (FBS) and 1% antibiotics. Put the nutrient mixture in a 75 T-flask. Then, put the T-flask in an incubator at 37° C. and with 5% CO2 for cell culture. When the growth of the cells reached 90% confluence, a cell subculture was performed.

2. Primary Dermal Fibroblast Cell Culture

Place the foreskin, provided by Dr. Wu, Nan-Lin from the Mackay Memorial Hospital in Hsinchu, Taiwan, in a solution of DMEM and 5% gentamycin, and store the solution at 4° C. Separate the foreskin from the solution, and then wash the foreskin by using phosphate-buffer saline (PBS) once and 1% antibiotics once. Place the foreskin in a 6 cm or 10 cm culture tray that contains a HBSS broth. The subcutaneous fat of the foreskin was then removed from the foreskin, and the foreskin was then cut into a 0.5 cm to 0.5 cm square cube. The cut foreskin was placed in a centrifuge tube that contains 0.25% trypsin (GIBCO) and HBSS, and the tube was stored at 4° C. for 24 hours. Next day, place the cut foreskin in a culture tray. The skin was peeled off by two tweezers. The hypoderma in the hypodermis of the cut foreskin was transferred into another culture tray. The hypodermal cells in the hypoderma were removed by using tweezers. Then, cut the processed foreskin into several pieces, and put them in a solution of 0.04% trypsin at 37° C. for 5 minutes. And, add an equal volume of DMEM that contains 10% FBS into the trypsin solution. The small pieces that precipitated in the bottom of the tray were removed. Then, the cell solution was centrifuged at 1100 rpm for 5 minutes. After the centrifuging operation, remove the clean solution, and leave the cells that precipitated in the bottom of the centrifuge tube. Then, the cells were suspended in a broth. Next, diversify the cells. If there are more cells, then use a T-75 culture tray to plate the cells. If there are not a lot of cells, then use a T-25 culture tray. Finally, place the culture tray in an incubator at 37° C. and with 5% CO₂. The broth was changed after 2 to 3 days. Then, a subculture was performed in the next step.

3. Subculture of Dermal Fibroblasts and Human Keratinocytes

The cells were washed twice by PBS. Mix a 5 ml solution that contains 0.5% Trypsin-EDTA (GIBCO) with the washed cells, and place the solution in an incubator at 37° C. and with 5% CO2 for 5 minutes. Through the microscope, make sure that the cells were separated. Then, add a broth that contains FBS to neutralize the effects of trypsin. The mixture of the cell solution was centrifuged in a centrifuge tube at 1100 rpm for 5 minutes. After the centrifugation, the supernatant was removed. And, leave the cells that precipitated in the bottom of the centrifuge tube. Use a broth to diversify the cells, and implant the cells on a flask. Put the flask in an incubator at 37° C. and with 5% CO₂. Then, the broth was changed after 2 to 3 days. After that, the broth was changed twice a week.

4. Western Blot Assay

Protein electrophoresis and western blot assay were used to analyze the intracellular AQP-3. 5×10⁵ cells were plated onto a 6-cm round culture tray. Add 2 ml DMEM-mixed broth that contains 10% FBS and 1% antibiotics in the culture tray. Place the culture tray in an incubator at 37° C. for 24 hours. After that, a pure DMEM broth that does not contain FBS was used to prohibit cell growth. After another 24 hours, add a mixed broth of 2 ml that contains 0.1% THF and a testing agent (the 18β-glycyrrhetinic acid derivative) of 3 μM, 10 μM, or 30 μM into the round culture tray that was later put in an incubator at 37° C. for 24 hours. After the treatment of the 18β-glycyrrhetinic acid derivative, the cells were transferred and collected on ice of 4° C. Each culture tray was washed twice through the PBS. The cells were dissolved in a radioimmunoprecipitation assay buffer (17 mM Tris-HCl, pH 7.4, 50 mM NaCl, 5 mM EDTA, 1 mM sodium fluoride, 1% Triton X-100, 1% sodium deoxycholate, 0.1% SDS, 1 mM sodium orthovanadate, 1 mM PMSF, and 1 μg/ml aprotinin and leupeptin, freshly prepared). Peel off the cells, and use ultrasonic to pulverize the cells. Then, the cell solution was centrifuged at 13,200 rpm for 10 minutes at 4° C. The supernatant was collected. Use the Pierce protein assay kit (Pierce, Rockford, Ill.) to measure the protein quantity. Use proteins of 20 μg to operate the electrophoresis of 10% SDS-polyacrylamide gel. Then, use a PVDF membrane to perform electroblot. After the electroblot, put the PVDF membranes into a TBS-T (Tris-buffered saline and 0.05% Tween 20) solution that contains 0.5% skim milk. Shake the container of the TBS-T solution for 1 hour to prevent non-specific binding. After that, the membranes were then washed three times by the TBS-T. And, each time took 10 minutes. Next, add primary antibodies (1:500 dilutions) into the membranes, and store the membranes at 4° C. overnight. After that, the membranes were washed three times by the TBS-T, and each time took 10 minutes. Then, add secondary antibodies into the membranes. Wait for 1 hour. Then, wash the membranes through the TBS-T three times, and each time took 10 minutes. Finally, add a developing agent so as to print an image in a film in the dark room.

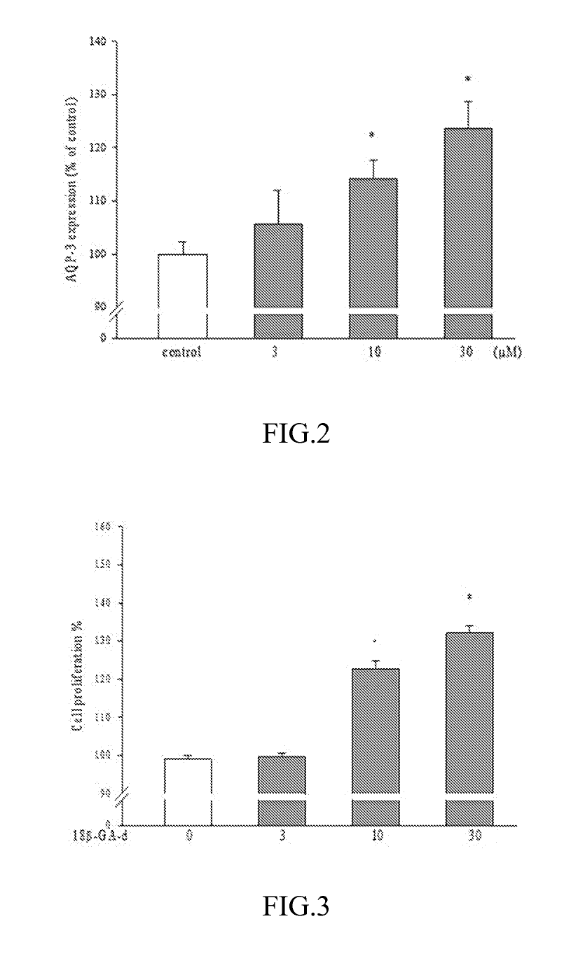

Recently, numerous studies have discussed the physiology of AQP-3 and wound healing, and the related molecular mechanisms. Hara Mariko et al. have reported that wound healing process was delayed in AQP-3 knockout mice; and Cong Cao has explored the effects of epidermal growth factor (EGF) on AQP-3 and wound healing. Cong Cao et al. have discovered, from the results of in vitro scratch wound healing assay, that expression of AQP-3 is significantly increased in EGF-treated groups, and wound healing was facilitated when treated with EGF. In summary, increasing the expression of AQP-3 is advantageous for skin wound healing. Therefore, the inventor(s) of the present invention further examined the correlation between the 18β-glycyrrhetinic acid derivative and human fibroblasts to see whether the 18β-glycyrrhetinic acid derivative can enhance the expression of AQP-3 in fibroblasts. The inventor(s) treated the fibroblasts by using an 18β-glycyrrhetinic acid derivative solution of 3 μM, 10 μM, or 30 μM. A tetrahydrofuran (THF) solution, which is used to treat 18β-glycyrrhetinic acid, was used as a control group. The western blot assay was applied for testing. According to the results of the testing, the treatment of 3 μM 18β-glycyrrhetinic acid derivative has no significant effects on AQP-3 expression, whereas the treatment of 10 μM or 30 μM 18β-glycyrrhetinic acid derivative can notably upregulate the expression of AQP-3. As shown in FIG. 2, 18β-glycyrrhetinic acid derivative can increase the AQP-3 concentration in fibroblasts up to 15-45%.

### Embodiment 3

### The Effects of 18β-Glycyrrhetinic Acid on Fibroblast Proliferation

Cell Proliferation Assay

1. MTT Assay

The cells were plated onto a 24-well culture plate, and each well evenly had the same concentration that is 3×10⁴ cells per well. Add a 500 μl DMEM-mixed broth that contains 10% FBS and 1% antibiotics. Place the broth in an incubator at 37° C. for 24 hours. When the growth of the cells reached 70-80% confluence, the cells were then washed twice through the PBS. After that, use a pure DMEM solution, which does not contain FBS, to stop the cell growth. Then, after 24 hours, the cells were washed twice through the PBS. Then, add into the 24-well culture plate a 300 μl solution that contains a broth of 0.1% THF or an 18β-glycyrrhetinic acid derivative-mixed broth of 3 μM, 10 μM, or 30 μM. Place the plate in an incubator at 37° C. for 24 hours. After that, take a picture of the sample for observing the situation of cell proliferation. Next, add into each well of the plate a 300 μl solution of MTT [3-(4,5-dimethylthiazol-2-yl)-2,5-diphenyl-2H-tetrazolium]. After 2 to 4 hours, cell proliferation was examined by measuring the light absorbability through an ELISA reader operated at a wave length of 550 nm

2. Trypan Blue Exclusion Assay

5×10⁵ cells were plated onto a 6-cm round culture tray. Add a 2 ml DMEM-mixed broth that contains 10% FBS and 1% antibiotics. Place the tray into an incubator at 37° C. for 24 hours. Wait for the cell growth to reach 70-80% confluence. The cells were then washed twice through the PBS. Use a pure DMEM broth that does not contain FBS to stop the cell growth. After 24 hours, the cells were washed twice through the PBS. Then, add into the 6-cm round culture tray a 2 ml broth that contains 0.1% THF, or a 2 ml 18β-glycyrrhetinic acid derivative-mixed broth that contains 18β-glycyrrhetinic acid derivative of 3 μM, 10 μM, or 30 μM. Place the tray in an incubator at 37° C. for 24 hours. Then, add into the tray a 2 ml solution of 0.5% Trypsin-EDTA. Place the tray in an incubator at 37° C. and with 5% CO₂ for 3-5 min. Use a microscope to see whether the cells are dissociated. After the cells were separated, a broth that contains FBS was added to the tray to neutralize the effects caused by Trypsin. Next, the cell-mixed solution was centrifuged in a centrifuge tube at 1,100 rpm for 5 minutes. After the centrifugation, remove the supernatant and leave the cells that precipitated in the tube bottom. Finally, use a broth to diversify the cells. Then, use a cell counter to count the number of cells in each sample.

Hara Mariko et al. proposed a theory in their study that upregulation of the AQP-3 expression can facilitate glycerol transport and promote cell proliferation. After it was evidenced that 18β-glycyrrhetinic acid derivative can significantly increase AQP-3 expression, we further tested whether 18β-glycyrrhetinic acid derivative can promote fibroblast proliferation. The effect of 18β-glycyrrhetinic acid derivative on fibroblast was examined using MTT assay. Our results showed that 18β-glycyrrhetinic acid derivative can notably induce fibroblast proliferation at the concentrations of 10 μM and 30 μM (FIG. 3), and that total cell number can increase up to 25˜85%.

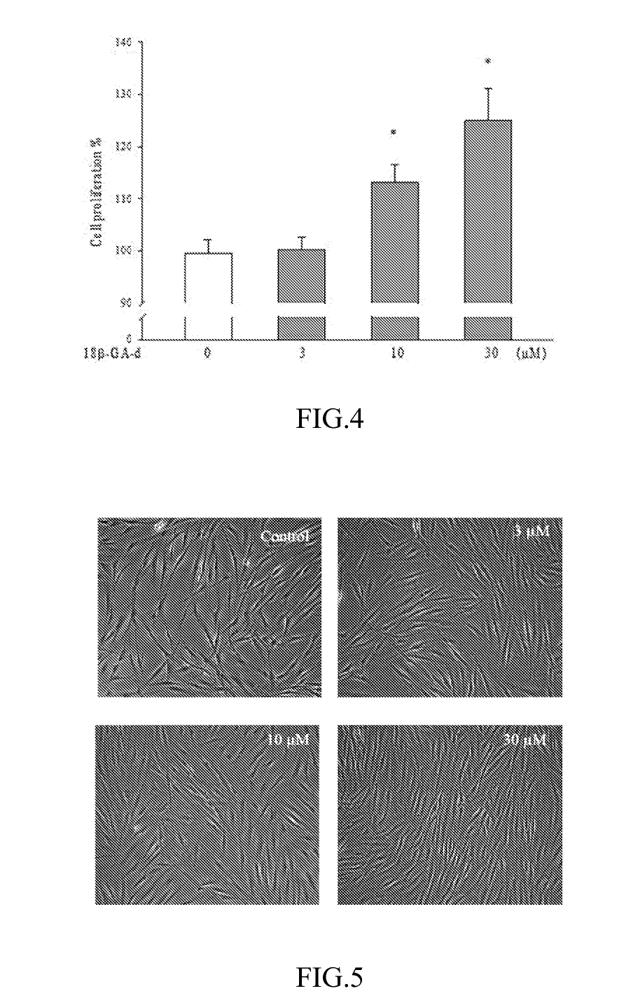

The principle of the MTT assay is measuring the activity of the reductase, which cuts the tetrazolium ring and reduces the yellow MTT dye in solution to insoluble purple formazan. The absorbance of the colored solution can be used to measure relative cell concentration. In order to prevent experimental errors resulted from reduced reductase activity which was caused by 18β-glycyrrhetinic acid derivative, another cell proliferation assay was used to cross examine the cell proliferation: Trypan blue exclusion assay. According to the results (FIG. 4), treatments of 10 μM and 30 μM 18β-glycyrrhetinic acid derivative indeed promote cell proliferation.

As shown in FIG. 5, fibroblast proliferation was also observed under a light microscope. Treatment groups of 10 μM and 30 μM 18β-glycyrrhetinic acid derivative have significantly more cells than untreated control groups. Given the results of FIG. 3, FIG. 4, and FIG. 5, 18β-glycyrrhetinic acid derivative at both 10 μM and 30 μM concentrations can promote fibroblast proliferation up to 25-85%.

### Embodiment 4

### The Effects of 18β-Glycyrrhetinic Acid on Migration of Fibroblasts

Cell Migration Assay

1. In Vitro Scratch Wound Healing Assay

5×10⁵ cells were plated into a 6-cm round culture tray. Add into the tray a 2 ml DMEM-mixed broth that contains 10% FBS and 1% antibiotics. Place the tray in an incubator at 37° C. for 24 hours. After that, replace the used broth with a pure DMEM broth that contains no FBS to stop the cell growth. After 24 hours, use a 200 μl tip to scratch the tray inside the tray. The scratch was considered a wound. Then, add into the tray a 2 ml broth that contains 0.1% THF, or a 2 ml 18β-glycyrrhetinic acid derivative-mixed broth that contains 18β-glycyrrhetinic acid derivative of 3 μM, 10 μM, or 30 μM. Place the tray in an incubator at 37° C. Take a picture of the wound-healing progress at the 0 hour, 6th hour, 12th hour, and 24th hour.

2. Electric Cell-Substrate Impedance Sensing (ECIS)

ECIS was used to examine the wound healing progress. Plate 7×10⁴ cells in an ECIS-specific culture tray. Add into the tray a 400 μl DMEM-mixed broth that contains 10% FBS and 1% antibiotics. Place the tray in an incubator at 37° C. for 24 hours (the culture tray was wetted for 1 hour before the incubation). Then, use an apparatus to examine the growth and homogeneity of the cells that were prepared the night before. After the cells become stable for a period of time (two hours), perform electric shock over the cells to damage them. Then, add into the tray a 400 μl broth that contains 0.1% THF, or a 400 μl 18β-glycyrrhetinic acid derivative-mixed broth that contains 18β-glycyrrhetinic acid derivative of 3 μM, 10 μM, or 30 μM. Place the tray in an incubator at 37° C. During the incubation, the impedance was monitored in real-time and recorded.

3. Statistics

Sigma-plot software was used to calculate mean±standard error (SE) as a representative value. An unpaired, two-tailed Student's t test was used for statistics, and p value less than 0.05 was considered significantly different, with an asterisk (*) as a note.

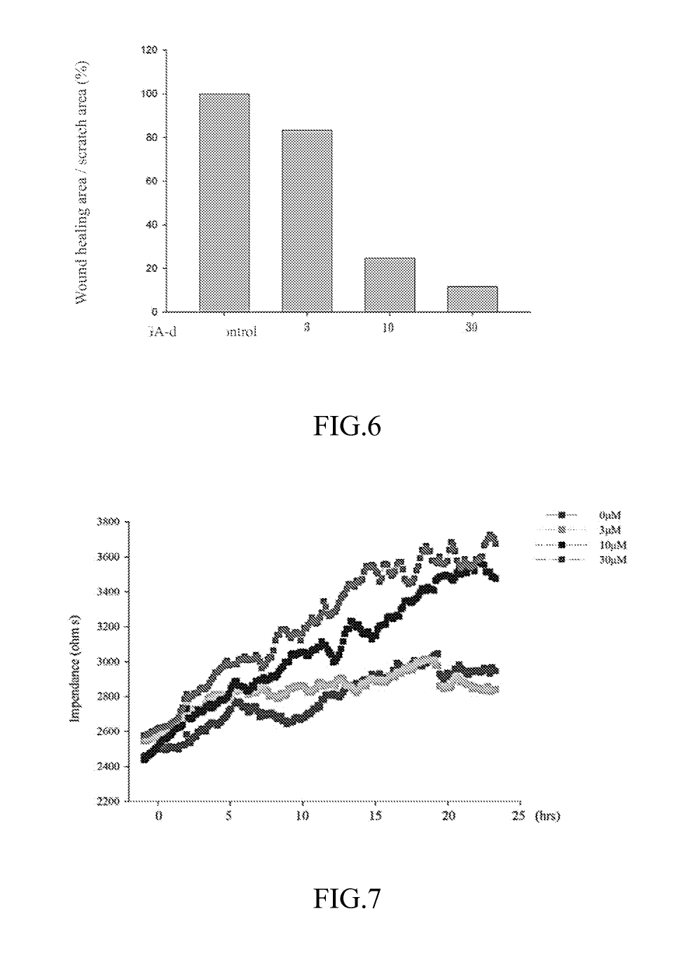

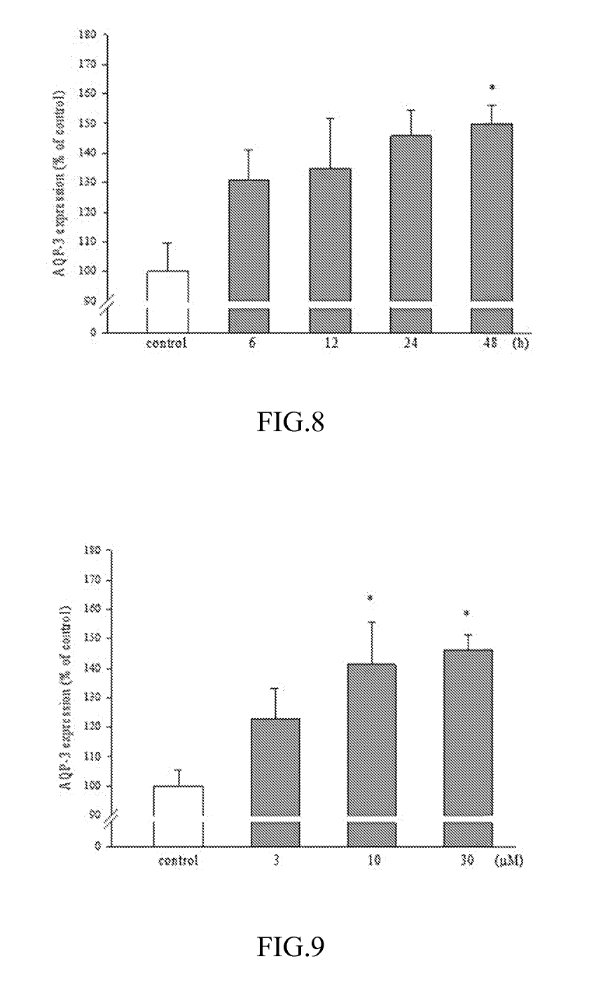

In their cell proliferation experiments, Hara Mariko et al. proposed a theory that upregulation of the AQP-3 expression can facilitate glycerol transport and promote cell proliferation. Meanwhile, the idea that activation of AQP-3 expression can facilitate water transport and enhance cell migration was also discussed. Hence, we examined cell migration by in vitro scratch wound healing assay. After the treatment with 3 μM, 10 μM, or 30 μM 18β-glycyrrhetinic acid derivative, under a microscope, cell migration was photographed at the 6th hour, 12th hour and 24th hour. Our results (FIG. 6) indicated that in the 6th-hour post-treatment, no cell migration of fibroblast was observed in either experimental or control groups. However, in the 12th-hour post-treatment, the cells in 10 μM and 30 μM 18β-glycyrrhetinic acid derivative treated groups begin to migrate toward the scratched wound. In the 24th-hour post-treatment, significant cell migration toward the scratched wound was observed in both groups, which suggested that 18β-glycyrrhetinic acid derivative can promote cell migration.

Following in vitro scratch Wound Healing assay, we further verified cell migration by using electric cell substrate impedance sensing (ECIS). ECIS measures the change in impedance of a small electrode to AC current flow. The resistance (impedance) positively correlates with cell densities. ECIS is different from scratch assay in that in ECIS, cells grow on the electrodes, and current flow damages the cells and cell density can be monitored in real time. Following treatments of 18β-glycyrrhetinic acid derivative, the impedance of 10 μM and 30 μM started and continued to increase, which suggested that the wound heals more rapidly (FIG. 7); and from the results of FIG. 6 and FIG. 7, 18β-glycyrrhetinic acid derivative can promote fibroblast cell migration.

HaCaT (Human Keratinocytes Cell Line)
(1) Examination of the effects of 18β-glycyrrhetinic acid derivative on AQP-3 expression

Following examination the activity and effects of 18β-glycyrrhetinic acid derivative on human fibroblast, we further tested the effects of 18β-Glycyrrhetinic acid derivative on AQP-3 in human keratinocytes. First, the cells that were treated with 30 μM 18β-glycyrrhetinic acid derivative were used as the experimental group, and the control group was treated with THF, the solvent for 18β-glycyrrhetinic acid derivative. The cells, which were collected in the 6th-hour, 12th-hour, and 24th-hour post-treatments, were analyzed by the western blot. According to the results, AQP-3 expression shows no significant increase at the 6th-hour and 12th-hour post-treatments. Yet, at the 24th-hour post-treatment, the increased expression of AQP-3 was observed, and reached the peak at the 48th-hour post-treatment. Subsequently, we examined the effects of various concentrations of 18β-glycyrrhetinic acid derivative on AQP-3. As demonstrated in our results, AQP-3 expression in human keratinocytes increased accordingly with increased concentrations of 18β-glycyrrhetinic acid derivative (FIG. 9).

Given the above, 18β-glycyrrhetinic acid derivative can indeed increase AQP-3 expression in human keratinocytes up to 45-65%.
(2) The effects of 18β-glycyrrhetinic acid derivative on cell proliferation in human keratinocytes.

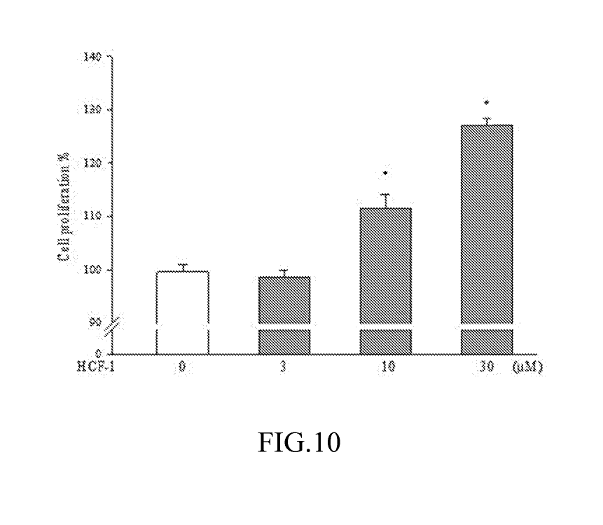

After it was demonstrated that 18β-glycyrrhetinic acid derivative can significantly increase AQP-3 expression in human keratinocytes, we also explored whether 18β-glycyrrhetinic acid derivative can promote cell proliferation. MTT assay was used to examine the effects of 18β-glycyrrhetinic acid derivative on human keratinocyte proliferation. The results indicated that 18β-glycyrrhetinic acid derivative, at the concentrations of 10 μM and 30 μM, can notably promote cell proliferation (FIG. 10), and human keratinocytes increased around 15˜45%.

The foregoing detailed descriptions are practical examples of the present invention. It should be noted, however, that such examples are provided for the purposes for demonstration rather than limitation. Applications of said compound in medicinal cosmetology are all included in the present invention. Many changes and modifications in the above described embodiments of the invention can, evidently, be carried out without departing from the scope thereof. Accordingly, to promote the progress in science and the useful arts, the invention is disclosed and is intended to be limited only by the scope of the appended claims.

What is claimed is:

1. A method for preventing diseases caused by aquaporin deficiency with a novel compound, wherein the novel compound is a 18β-glycyrrhetinic acid derivative, the chemical structure of which is shown in FIG. 1, wherein R is selected from one of the following functional groups: H, CH₃, CH(CH₃)₂, and CH₂Ph.

2. The method as claimed in claim 1, wherein the 18β-glycyrrhetinic acid derivative is a pharmaceutically-acceptable salt of 18β-glycyrrhetinic acid.

3. The method as claimed in claim 1, wherein the 18β-glycyrrhetinic acid derivative is a solvate of 18β-glycyrrhetinic acid.

4. The method as claimed in claim 1, wherein the 18β-glycyrrhetinic acid derivative is a pharmaceutically active derivative of 18β-glycyrrhetinic acid.

5. The method as claimed in claim 1, wherein the novel compound can increase AQP-3 expression in fibroblasts.

6. The method as claimed in claim 1, wherein the novel compound can increase AQP-3 expression in human keratinocytes.

7. The method as claimed in claim 1, wherein the novel compound is used for glycerol transport.

8. The method as claimed in claim 1, wherein the novel compound is used to increase the number of fibroblast.

9. The method as claimed in claim 1, wherein the novel compound is used to increase the number of human keratinocytes.

10. The method as claimed in claim 1, wherein the novel compound is used for wound healing.

11. A method for preventing diseases caused by aquaporin deficiency with a medicinal composition, wherein the medicinal composition comprises of: a 18β-glycyrrhetinic acid derivative, the chemical structure of which is shown in FIG. 1, wherein R is selected from one of the following functional groups: H, CH₃, CH(CH₃)₂, and CH₂Ph; and at least one medicinally-acceptable carrier.

12. The method as claimed in claim 11, wherein the medicinally-acceptable carrier is a diluent.

13. The method as claimed in claim 11, wherein the medicinally-acceptable carrier is a filler.

14. The method as claimed in claim 11, wherein the medicinally-acceptable carrier is a binder.

15. The method as claimed in claim 11, wherein the medicinally-acceptable carrier is a disintegrating agent.

16. The method as claimed in claim 11, wherein the medicinally-acceptable carrier is a lubricant.
